(19) United States
(12) Patent Application Publication
(10) Pub. No.: US 2016/0296177 A1
(43) Pub. Date: Oct. 13, 2016

(54) CARDIAC ARREST ALERT SYSTEM AND METHOD

(71) Applicant: CardioThrive, Inc., Concord, CA (US)

(72) Inventor: Peter D. Gray, Vallejo, CA (US)

(21) Appl. No.: 14/685,173

(22) Filed: Apr. 13, 2015

(51) Int. Cl. A61B 5/00; G08B 21/02; A61B 5/0452

(52) U.S. Cl. CPC A61B 5/746; A61B 5/0452; G08B 21/02

(57) ABSTRACT

A cardiac arrest alert system and method are provided that can identify the onset of a Sudden Cardiac Arrest and that, via audio, visual and electronic alerts, can directly inform a rescuer of the need to get a defibrillator to the victim's location as rapidly as possible. In one embodiment, the system can be used in conjunction with a specifically paired portable AED which can then emit audio and visual alerts that its services are required by the SCA victim. In other embodiments, the system makes use of a subcutaneous sensor component which is paired with and linked to an external monitoring/receiver device that emits the audio and visual alerts in addition to alerting EMS with the location.

### FIELD

The disclosure relates generally to methods and arrangements relating to medical devices. More specifically, the disclosure relates to the systems and methods used in a cardiac arrest alert system and in a preferred embodiment to a cardiac arrest alert system with a subcutaneous sensor system intended to identify the actual or imminent onset of a Sudden Cardiac Arrest (SCA) especially where this may be used to alert the carrier of a nearby portable Automated External Defibrillator (AED), EMS, companions or bystanders in order to more rapidly save the life of the Sudden Cardiac Arrest victim.

### BACKGROUND

A primary task of the heart is to pump oxygenated, nutrient-rich blood throughout the body. Electrical impulses generated by a portion of the heart regulate the pumping cycle. When the electrical impulses follow a regular and consistent pattern, the heart functions normally and the pumping of blood is optimized. When the electrical impulses of the heart are disrupted (i.e., cardiac arrhythmia), this pattern of electrical impulses becomes chaotic or overly rapid, and a Sudden Cardiac Arrest may take place, which inhibits the circulation of blood. As a result, the brain and other critical organs are deprived of nutrients and oxygen. A person experiencing Sudden Cardiac Arrest may suddenly lose consciousness and die shortly thereafter if left untreated.

The most successful therapy for Sudden Cardiac Arrest is prompt and appropriate defibrillation. A defibrillator uses electrical shocks to restore the proper functioning of the heart.

A crucial component of the success or failure of defibrillation, however, is time. Ideally, a victim should be defibrillated immediately upon suffering a Sudden Cardiac Arrest, as the victim's chances of survival dwindle rapidly for every minute without treatment.

One of the biggest challenges with saving victims of a Sudden Cardiac Arrest is that they are often not near an external defibrillator when it happens and that once EMS are actually notified it then takes the medical professionals too long to arrive at the scene with a defibrillator to be able to successfully revive the victim. In many cases the SCA is not even witnessed when the person experiences the Sudden Cardiac Arrest and this means that they are not found for some period of time and then, once they are found, it is already too late to successfully resuscitate them.

There are a wide variety of defibrillators. For example, Implantable Cardioverter-Defibrillators (ICD) involve surgically implanting wire coils and a generator device within a person. ICDs are typically for people at high risk for a cardiac arrhythmia. When a cardiac arrhythmia is detected, a current is automatically passed through the heart of the user with little or no intervention by a third party.

Another, more common type of defibrillator is the automated external defibrillator (AED). Rather than being implanted, the AED is an external device used by a third party to resuscitate a person who has suffered from sudden cardiac arrest. FIG. 1 illustrates a conventional AED 100, which includes a base unit 102 and two pads 104. Sometimes paddles with handles are used instead of the pads 104. The pads 104 are connected to the base unit 102 using electrical cables 106.

A typical protocol for using the AED 100 is as follows. Initially, the person who has suffered from sudden cardiac arrest is placed on the floor. Clothing is removed to reveal the person's chest 108. The pads 104 are applied to appropriate locations on the chest 108, as illustrated in FIG. 1. The electrical system within the base unit 102 generates a high voltage between the two pads 104, which delivers an electrical shock to the person. Ideally, the shock restores a normal cardiac rhythm. In some cases, multiple shocks are required.

Although existing technologies work well, there are continuing efforts to improve the effectiveness, safety and usability of automatic external defibrillators. Accordingly, efforts have been made to improve the availability of automated external defibrillators (AED), so that they are more likely to be in the vicinity of Sudden Cardiac Arrest victims. Advances in medical technology have reduced the cost and size of automated external defibrillators (AED). Some modern AEDs approximate the size of a laptop computer or backpack. Even small devices may typically weigh 4-10 pounds or more. Accordingly, they are increasingly found mounted on the walls in public facilities (e.g., airports, schools, gyms, etc.) and, more rarely, residences. Unfortunately, the average survival rates for an out-of-hospital Sudden Cardiac Arrest remain abysmally low (around 8.3%).

Yet such wall-mounted solutions, while efficacious, are still less than ideal for most real world situations. Assume, for example, that a person suffers from a cardiac arrest in an airport in which multiple AEDs have been distributed. The victim's companion, or a bystander, would nevertheless have to locate and run towards the nearest AED, pull the device out of the locked cabinet on the wall, and then return to the collapsed victim before they can even begin to render assistance. During that time, precious minutes may have passed. According to some estimates, the chance of surviving a sudden cardiac arrest is 90% if the victim is defibrillated within one minute, but declines by 10% for every minute thereafter.

An additional challenge is that a sudden cardiac arrest may take place anywhere. People often spend time away from public facilities and their homes. For example, a sudden cardiac arrest could strike someone while biking in the hills, skiing on the mountains, strolling along the beach, or jogging on a dirt trail. Ideally, an improved AED design would be compact, light, and resistant to the elements and easily attached or detached from one's body. The typical AED design illustrated in FIG. 1, which includes a sizable console or power unit whose form factor is similar to that of a laptop or backpack, seems less than ideal for the outdoors and other rigorous environments.

New and improved designs are allowing AEDs to become ultra-portable and hence to able to be easily carried by an at-risk person as they go about all of their daily activities and thus are able to be close at hand when a sudden cardiac arrest strikes outside of a hospital environment or a high traffic public area with a Public Access Defibrillator. There are also improvements being made in the area of device usability and ease of operation for untrained bystanders. As noted above, every minute of delay or distraction can substantially decrease the victim's probability of survival.

Another type of defibrillator is the Wearable Cardioverter Defibrillator (WCD). Rather than a device being implanted into a person at-risk from Sudden Cardiac Arrest, or being used by a bystander once a person has already collapsed from experiencing a Sudden Cardiac Arrest, the WCD is an external device worn by an at-risk person which continuously monitors their heart rhythm to identify the occurrence of an arrhythmia, to then correctly identify the type of arrhythmia involved and then to automatically apply the therapeutic action required for the type of arrhythmia identified, whether this be cardioversion or defibrillation. These devices are most frequently used for patients who have been identified as potentially requiring an ICD and to effectively protect them during the two to six month medical evaluation period before a final decision is made and they are officially cleared for, or denied, an ICD.

While these devices are worn by the patient and monitor them almost 24 hours per day, they are only worn by patients that have been diagnosed as needing an ICD or its equivalent. This constant monitoring is not available to other types of patient, and almost 50% of all Sudden Cardiac Arrests happen to people that have had no prior diagnosis of cardiac problems.

There are multiple types of cardiac monitoring technologies on the market today. These vary from traditional holters and event recorders (including the latest event recorder patches and subcutaneous loop recorders), to mobile cardiac monitoring devices and to close-to-real-time ambulatory telemetry devices. All of these are intended to provide accurate data which can aid in the accurate medical diagnoses of patients under the scrutiny/care of medical professionals and they are not intended to provide any sort of emergency services. The medical staff at a central monitoring center may, upon witnessing a suspected syncopy event in the ECG strip of an ambulatory patient being monitored, then call the patient to find out if they are conscious and if not then they may escalate the call to the patient's family, doctor and even EMS—but this is not intended to identify lethal arrhythmias such as Ventricular Fibrillation and Ventricular Tachycardia, or any other life threatening cardiac rhythm that requires defibrillation, and to assist in getting a life saving defibrillator to the victim as fast as possible.

Cardiac monitoring devices are intended for the detection of non-lethal arrhythmias to aid in physician diagnoses. They are found in a range of sizes, embody a range of technologies and a range of approaches (from recording the ECG for later analysis which is then provided in a report to the clinician, to almost-real-time telemetry systems). These do not constitute an automated cardiac arrest alert system, as they are not intended to detect a lethal arrhythmia and trigger an alarm or alert.

ICDs and WCDs are complex and expensive devices that both automatically detect and automatically treat the lethal arrhythmias of Ventricular Fibrillation and Ventricular Tachycardia, but only for patients who have already been diagnosed as being very sick and who are known to be at a high risk of imminent cardiac arrest. These are not considered to be clinically appropriate for prescription to patients that do not (yet) meet the strict definitions for being at high risk, nor are they approved for reimbursement by the insurers for such patients.

WCDs currently experience the challenge of reduced accuracy in identifying lethal arrhythmias, as the ECG signals that are obtained from skin mounted ECG sensors are fainter, noisier and subject to much more interference from patient motion-triggered artifacts, and as such result in a higher level of false positives. For this reason WCDs currently incorporate a patient-controlled override button, allowing the patient the ability to prevent the WCD from shocking them inappropriately in the event of a false positive.

One of the reasons that the national annual mortality burden from SCA remains so high is that there is no system for immediate notification that a patient has experienced an Out Of Hospital Cardiac Arrest. Such a system would enable a focused and rapid response and a significantly increased likelihood of successful rescucitation and hence survival.

### BRIEF DESCRIPTION OF THE DRAWINGS

FIG. 1 diagrammatically illustrates an example of a conventional external defibrillator;

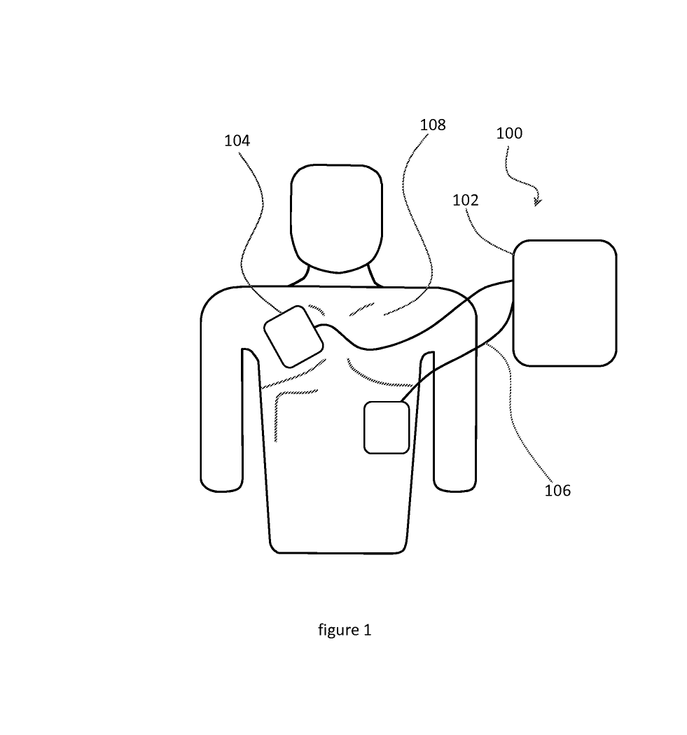

FIG. 2 illustrates an example of an implementation of a cardiac arrest alert system;

FIG. 3A illustrates the sensor system in a block diagram that may be part of the cardiac arrest alert system;

FIG. 3B illustrates the receiver system in a block diagram that may be part of the cardiac arrest alert system;

FIG. 4 illustrates an example of a subcutaneous sensor component;

FIG. 5 illustrates an example of a skin mounted patch sensor component;

FIG. 6 illustrates examples of an external monitoring base station;

FIG. 7 illustrates an example of an external monitoring watch;

FIG. 8 illustrates an example of an external monitoring cellular phone;

FIG. 9 illustrates an example of an external monitoring portable AED;

FIG. 10 illustrates the interaction of system components in a block diagram; and

FIG. 11 illustrates the range of locations on a patient's body that the sensor system can be placed.

### DETAILED DESCRIPTION OF ONE OR MORE EMBODIMENTS

The disclosed system and method are a cardiac arrest alert system that can identify the onset of a Sudden Cardiac Arrest and that, via audio, visual and electronic alerts, can directly inform a rescuer of the need to get a defibrillator to the victim's location as rapidly as possible. In doing so, the system aids in reducing the victim's effective time to first shock defibrillation.

This device, by notifying bystanders, key companions or family, and also EMS, to the occurrence and location of a cardiac arrest, and hence the urgent need for immediate defibrillation of the SCA victim, will save more lives than the current methodology that relies primarily on random chance that there are witnesses to both observe the victim's collapse and also to recognize that the victim has been struck by a SCA event. The system and method ensure that an actionable alert occurs even if the SCA happens while the victim is sleeping—which is currently a scenario that almost guarantees the death of the victim—and in this manner alone it significantly increases the likelihood that an external defibrillator can be applied to the victim in time to resuscitate them. The system and method ensure that, even if the victim has a SCA event while alone and out of sight of anyone else, the relevant people are immediately notified of the emergency and of the victim's location and they are then able to take the appropriate actions.

In one embodiment, the invention can even be used in conjunction with a specifically paired portable AED which can then emit audio and visual alerts that its services are required by the SCA victim. Such audio and visual alerts by the AED can also help in ensuring that the AED is located as rapidly as possible so that it can be used on the SCA victim with the minimum of delay. In another embodiment, the system and method makes use of a subcutaneous sensor component which is paired with and linked to an external monitoring/receiver device that emits the audio and visual alerts in addition to alerting EMS with the physical location of the patient. In another embodiment, the system and method makes use of a skin mounted disposable patch sensor component that is paired with and linked to an external monitoring/receiver device that emits the audio and visual alerts in addition to alerting EMS with the location. In any of these embodiments the system is designed to perform periodic self checks with regards to the energy source power levels and the operational health and readiness of the system. The results of which are reported through the alert/alarm component 206.

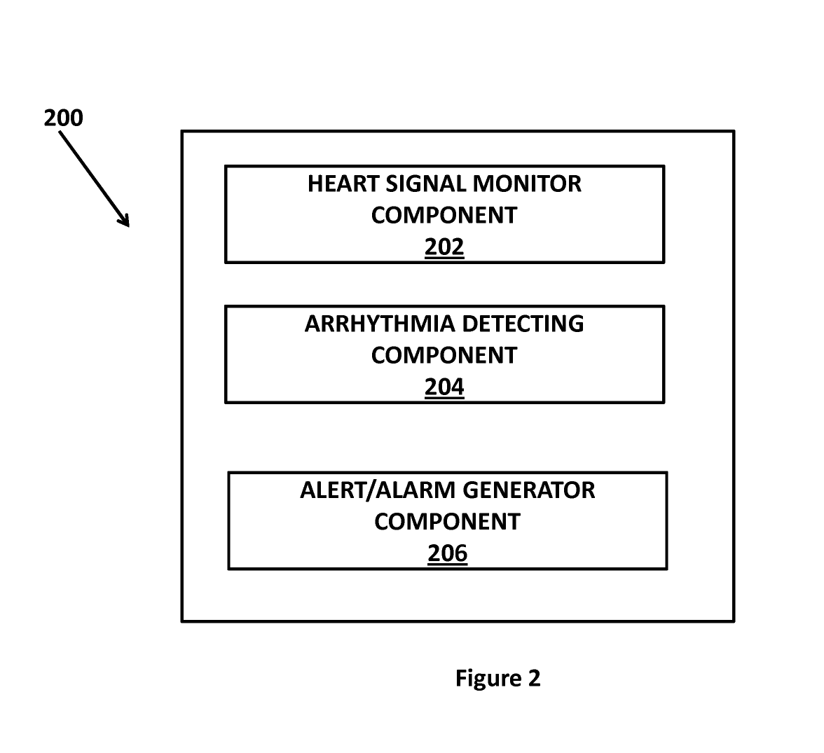

FIG. 2 illustrates an example of an implementation of a cardiac arrest alert system 200 that may be used for the above purposes. The system 200 may have a heart signal monitor component 202, an arrhythmia detecting component 204 and an alert/alarm component 206 in which each of these components is coupled together. The system may be a piece of hardware, but each component 202-206 may be implemented in hardware, software or a combination of hardware and software as would be understood be those skilled in the art. The disclosure however, is not limited to any particular implementation of these components.

The heart signal monitor component 202 may monitor's a patient's heart signals, such as an ECG, and feed the captured heart signal data to the arrhythmia detecting component 204 that detects whether there is a lethal arrhythmia within the heart signals. In some embodiments, the heart signal monitor component 202 may receive more than one ECG signal from the patient and the heart signal monitor component 202 may monitor and analyze and compare/contrast the at least more than one different ECG signals from the same patient and selects the most accurate or reliable ECG signal.

If a lethal arrhythmia is detected, the alert/alarm component 206 may generate an alert/alarm about the lethal arrhythmia (to various people, systems, etc.) so that defibrillation may be applied to the patient. The alert/alarm component 206 may generate, or cause to be generated, an audio alarm, a visual alarm, a vibration alarm or an electronic or data alarm.

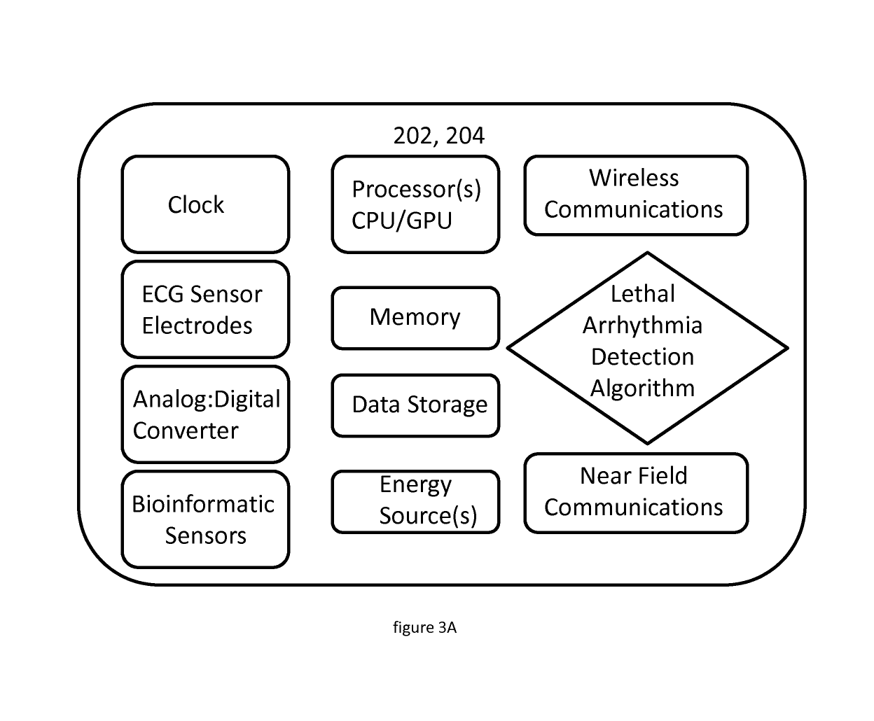

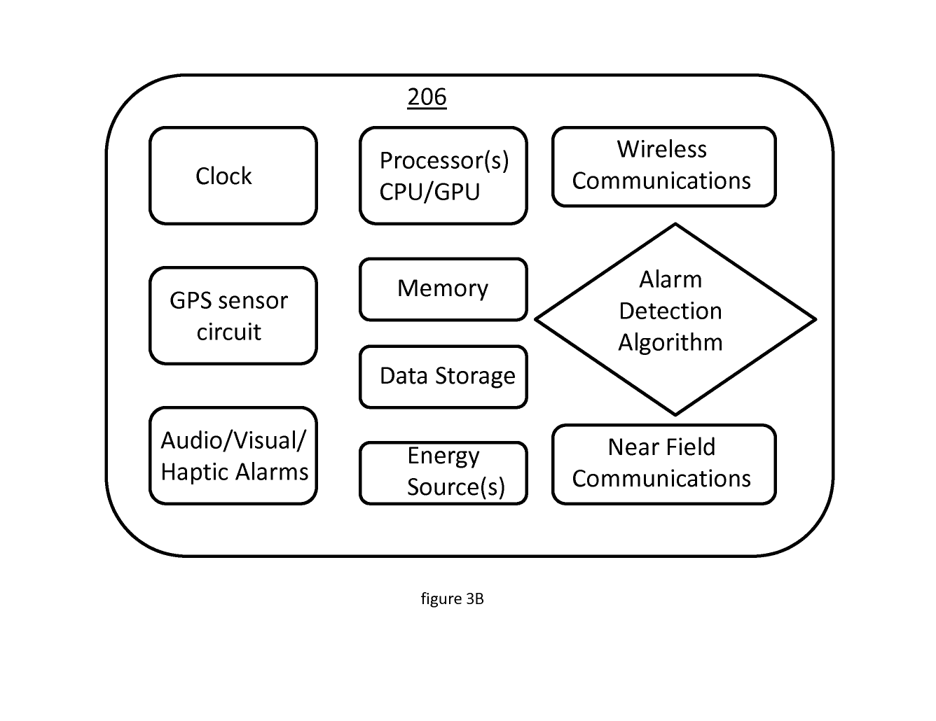

FIG. 3A shows a block diagram of a sensor function for the disclosed system with further details of the heart signal monitor component 202 and the arrhythmia detecting component 204. The sensor function may, in one implementation, passively monitor the patient's ECG looking for a lethal arrhythmia (such as Ventricular Fibrillation, Ventricular Tachycardia, Pulseless Electrical Activity, and Asystole). This is much like a smoke alarm looks for smoke, and then produces and communicates an alarm upon detecting smoke. Here the sensor function looks for a defined type of lethal arrhythmia and then produces and communicates an alarm upon detecting the defined type of lethal arrhythmia. In one embodiment, the analog signal from the ECG sensor(s) is passed through an analog to digital converter and then the resulting digital signal is then analyzed by a lethal arrhythmia detection algorithm which seeks to identify a lethal arrhythmia while running on the processor and memory as shown in FIG. 3A. In another embodiment the analog signal is processed directly without first passing through an analog to digital converter. Once a lethal arrhythmia is detected then the sensor function uses its communications circuitry (wireless communications circuits shown in FIG. 3A) to send out an alert or notification to the receiver function shown in FIG. 3B.

In one embodiment the receiver function may be combined into the same hardware as the sensor function. In another embodiment, the two functions are found in separate pieces of hardware. In some embodiments the patient has multiple sensor function devices placed in different locations about their body, which can include skin surface as well as subcutaneous and implanted locations. This ensures that the system has the maximum opportunity for eliminating noise and motion artifacts for the ECG signals, as well as the ability to compare the analyzed ECG signals to other bioinformatic sensor signals such as physical pulse readings and impedance readings in order to double check the algorithmic conclusions.

FIG. 3B shows a block diagram of a receiver function, which passively listens for the alert or notification from the sensor function and, upon finding one, communicates with bystanders via various suitable audio and visual prompts, communicates its GPS (or another system's equivalent specification of geographic location and within building/structure location) location to Emergency Medical Services notifying them of a Sudden Cardiac Arrest, and triggers an alert to listed companions or family members via various forms of electronic communication. The receiver function can be embodied in many different forms: from a dedicated device that is carried about the patient's person, to a multi-function device such as a smart watch or other piece of smart/functional jewelry, to a fitness tracking or health tracking wearable, to a smart phone or tablet, or a dedicated medical monitoring device or a general purpose base station/gateway for the home or office or even for a healthcare provider/long term care facility. In some embodiments the patient's receiver function device is specifically paired via unique identifiers with the patient's sensor function device(s) to ensure that other signals or interference cannot somehow trigger a false alarm. In some embodiments the patient's receiver function device is linked to more than one sensor function devices on the patient, in order to ensure the best possible signal(s) and to ensure that there is both redundancy and cross-checking built into the system as well as maximum accuracy and the minimal chance for a false positive.

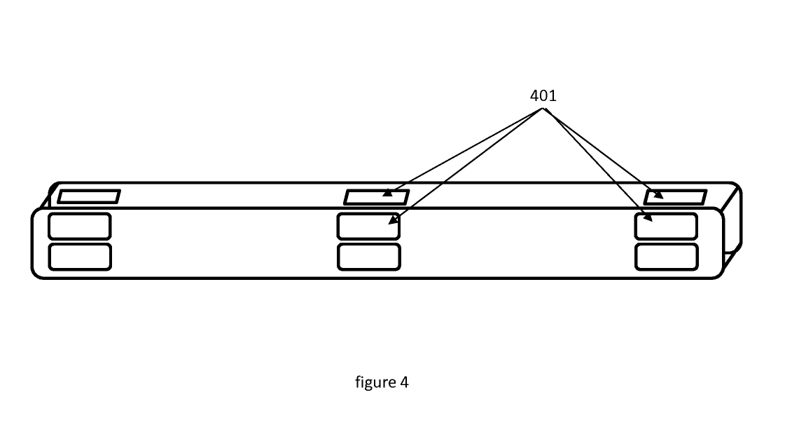

FIG. 4 shows an example of a miniature device suitable for subcutaneous placement in a patient or for full implantation in a patient. The exterior of the device consists of biocompatible materials, both conductive and non-conductive in nature. In some embodiments this device consists solely of the sensor function for the system which works in tandem with the separate receiver function outside of the patient's body. In other embodiments this device consists of the combined sensor and receiver functions and it communicates wirelessly (these subsystems are well known in the art) directly from its location within the patient's body. ECG sensors (401) are well known in the art and are located across the exterior of the device, in addition to additional bioinformatic sensors of other types. In some embodiments the device is solely powered by an internal battery. In other embodiments the device also utilizes any number of energy harvesting techniques (which are well known and understood in the art) such as those derived from the motion of the patient themselves or else wireless charging technologies (one example of which is the WiTricity system)

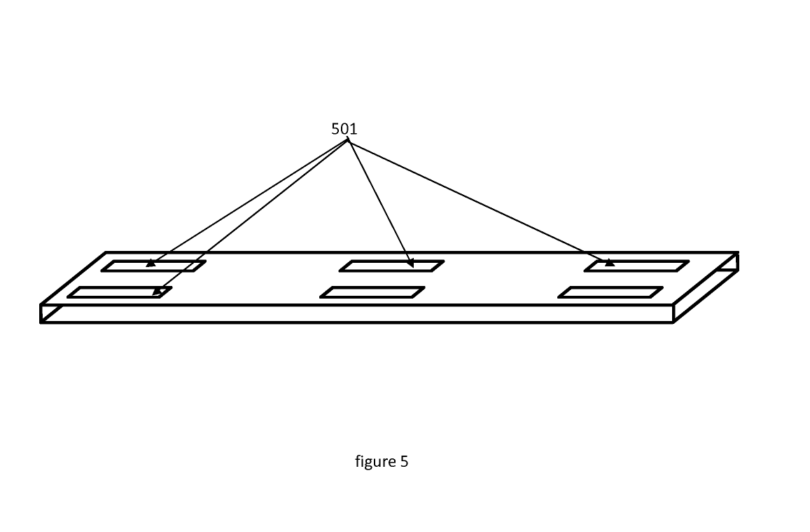

FIG. 5 shows shows a miniature device suitable for surface placement on the skin of a patient. In some embodiments this device consists solely of the sensor function for the system which works in tandem with the separate receiver function outside of the patient's body. In other embodiments this device consists of the combined sensor and receiver functions and it communicates wirelessly directly from its location on the surface of the patient's body. ECG sensors (501) are located across the patient-facing exterior surface of the device, in addition to additional bioinformatic sensors of other types. In some embodiments the device is solely powered by an internal battery. In other embodiments the device also utilizes any number of energy harvesting techniques (which are well known and understood) such as those derived from the motion of the patient themselves or else wireless charging technologies (one example of which is the WiTricity system)

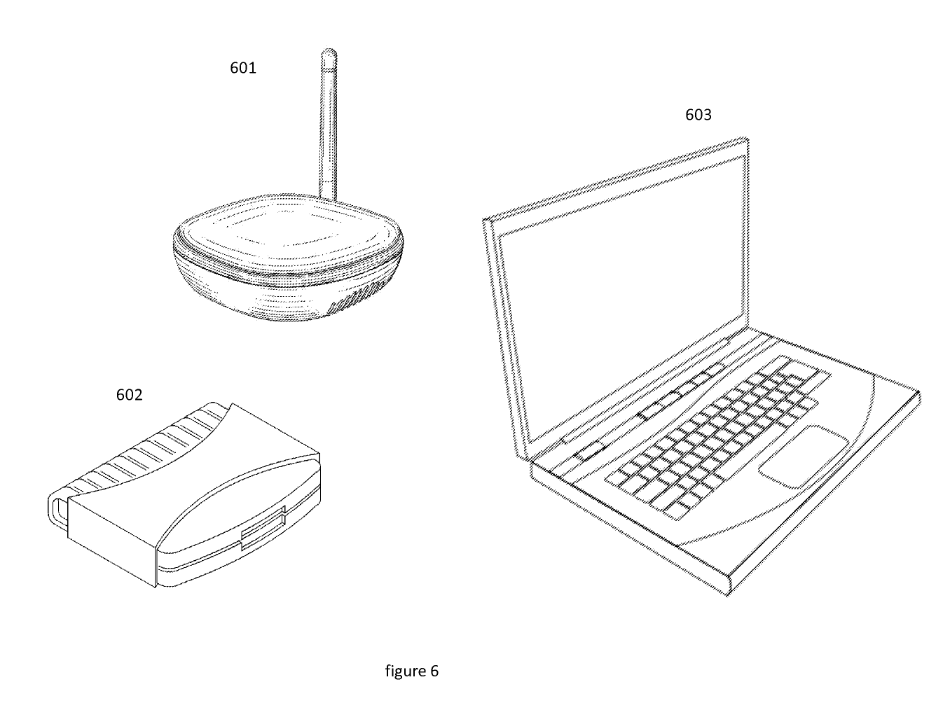

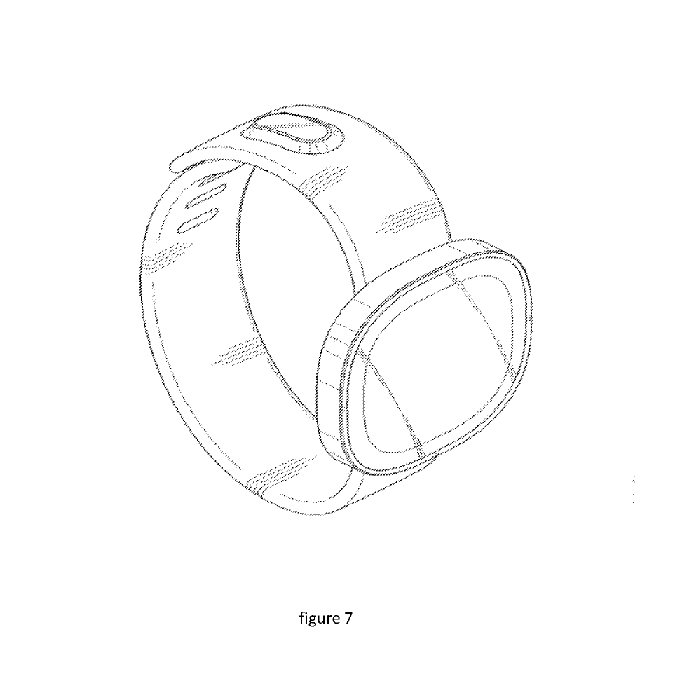

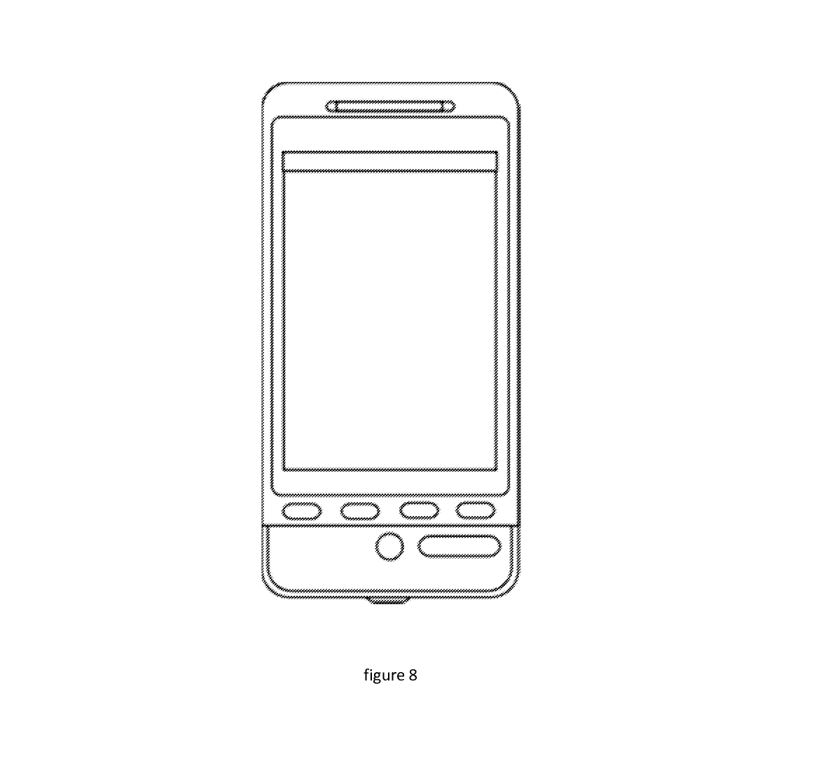

FIG. 6 shows examples (601, 602, 603) of several different types of dedicated or general purpose external base stations which fulfill the receiver system function and work with the patient's sensor(s), several embodiments include external base stations being built/incorporated directly into vehicles including mass transit systems, and buildings or infrastructure. FIG. 7 shows an example of an external monitoring smart watch. FIG. 8 shows an example of an external monitoring cellular/smart phone.

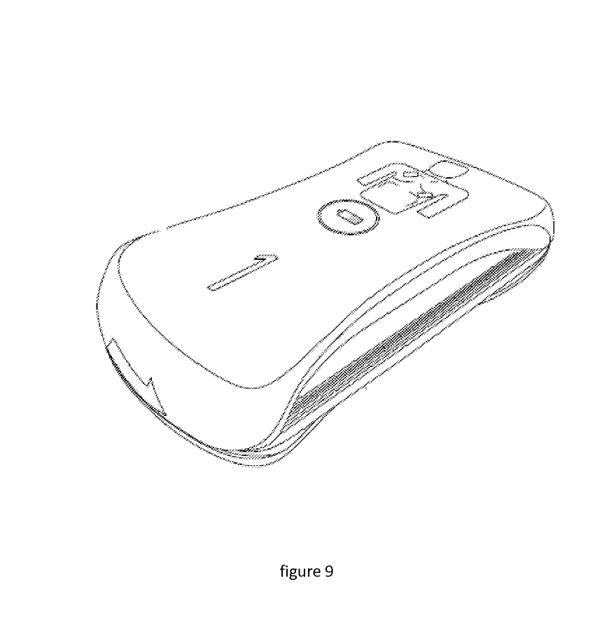

FIG. 9 shows an example of an external monitoring ultraportable AED, which fulfills the receiver function and works with the patient's sensor(s). In addition, it is an ideal companion device as it ensures the immediate/rapid availability of defibrillation in the event that a shockable lethal arrhythmia is detected.

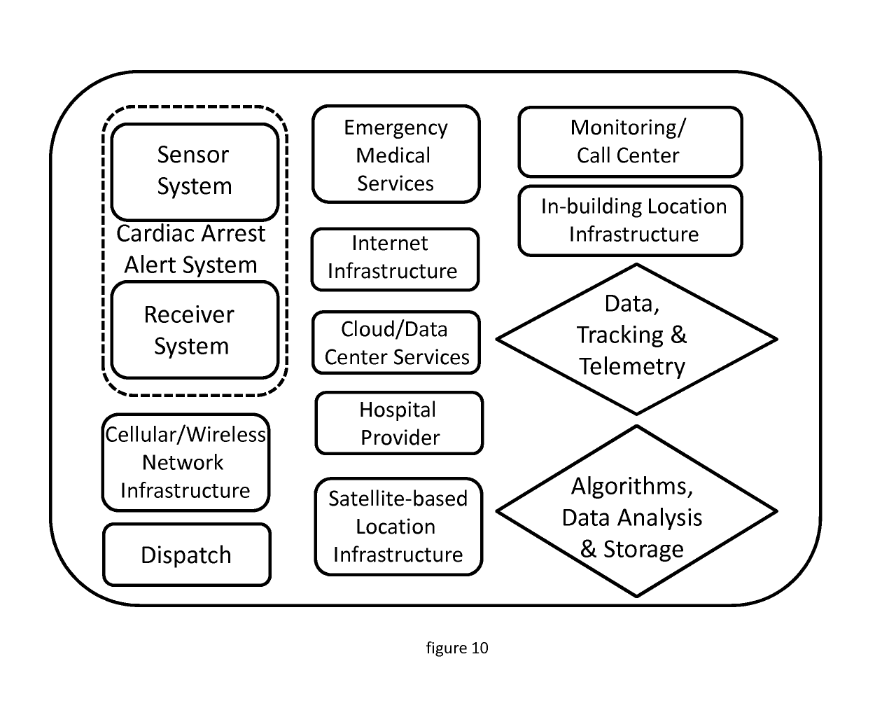

FIG. 10 shows the interactions of the disclosed system within the larger ecosystem, and highlights the other relevant infrastructures, in a block diagram. In some embodiments this includes interacting with and providing data to partially/fully automated monitoring and analysis systems.

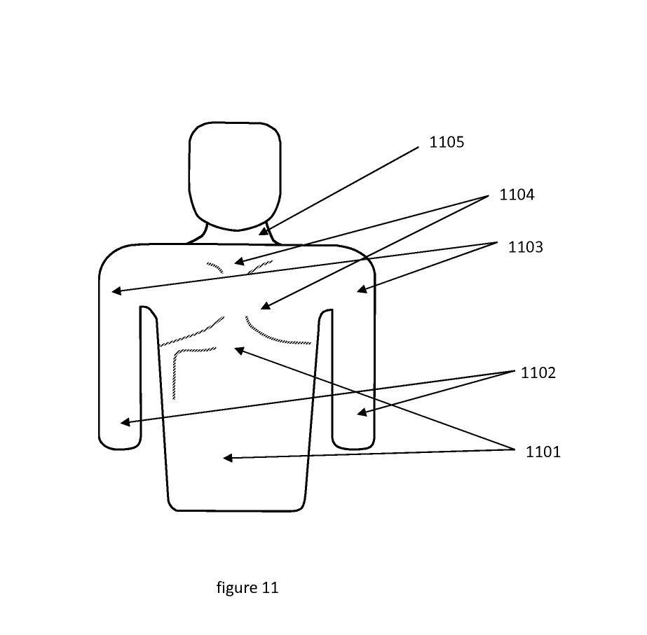

FIG. 11 shows many different examples (1101, 1102, 1103, 1104, 1105) of where one or more sensors can be located on, or just under, the skin of a patient, or else inside a patient.

While the foregoing has been with reference to a particular embodiment of the invention, it will be appreciated by those skilled in the art that changes in this embodiment may be made without departing from the principles and spirit of the disclosure, the scope of which is defined by the appended claims.

1. A cardiac arrest alert system, comprising:
a first subsystem that receives a patient's ECG signal;
a second subsystem that identifies a lethal arrhythmia based on the ECG signal of the patient; and
a third subsystem that generates and communicates an alarm when the lethal arrhythmia is identified in the ECG signal of the patient.

2. The alert system of claim 1, wherein the first subsystem further comprises a single lead ECG signal directly from one or more ECG sensors.

3. The alert system of claim 1, wherein the first subsystem further comprises three or more lead ECG signals directly from the ECG sensors.

4. The alert system of of claim 1, wherein the first subsystem further comprises at least one or more separate ECG sensors, and at least one or more separate ECG signal receivers.

5. The alert system of claim 1, wherein the first subsystem receives the ECG signal from at least one ECG sensing device.

6. The alert system of claim 5, wherein the first subsystem monitors and analyzes and compares/contrasts the at least more than one different ECG signals from the same patient and selects the most accurate or reliable.

7. The alert system of claim 1, wherein the second subsystem identifies at least Ventricular Fibrillation, Ventricular Tachycardia, Pulseless Electrical Activity and Asystole as lethal arrhythmias.

8. The alert system of claim 1, wherein the third subsystem generates, or causes to be generated, at least one of an audio alarm, a visual alarm, a vibration alarm and an electronic or data alarm.

9. The alert system of claim 8, wherein the generated alarm is communicated via wireless electronic means, directly or indirectly, to one or more additional devices which may then communicate their own alarms in any of the aforementioned forms to other people or devices.

10. The alert system of claim 1, wherein the alarm generation and communication subsystem generates, or causes to be generated, an alarm to be communicated to one or more of: bystanders, Emergency Medical Services, companions, medical professionals, family members, automated monitoring and data collection systems and records.

11. The alert system of claim 10, wherein the communicated alarm includes additional information on the physical location of the patient.

12. The alert system of claim 1, wherein the hardware and software includes a self checking/monitoring and reporting capability.

13. A method, comprising:
receiving a patient's ECG signal;
identifying a lethal arrhythmia based on the ECG signal of the patient; and
generating and communicating an alarm when a lethal arrhythmia is identified in the ECG signal of the patient.

14. The method of claim 13, wherein receiving the ECG signal further comprises receiving a single lead ECG signal directly from one or more ECG sensors.

15. The method of claim 13, wherein receiving the ECG signal further comprises receiving three or more lead ECG signals directly from the ECG sensors.

16. The method of claim 13, wherein receiving the ECG signal further comprises receiving the ECG signal from the at least one ECG sensing device.

17. The method of claim 16, wherein receiving the ECG signal further comprises monitoring, analyzing and comparing the at least more than one different ECG signals from the same patient and selecting the most accurate or reliable.

18. The method of claim 13, wherein identifying the lethal arrhythmia further comprises indentifying one of Ventricular Fibrillation, Ventricular Tachycardia, Pulseless Electrical Activity and Asystole.

19. The method claim 13, wherein generating the alarm further comprises generating at least one of an audio alarm, a visual alarm, a vibration alarm and an electronic or data alarm.

20. The method of claim 19 further comprising communicating the generated alarm via wireless electronic means, directly or indirectly, to one or more additional devices which may then communicate their own alarms in any of the aforementioned forms to other people or devices.

21. The method of claim 13, wherein communicating the alarm further comprising communicating the alarm to one or more of: bystanders, Emergency Medical Services, companions, medical professionals, family members, automated monitoring and data collection systems and records.

22. The method of claim 21, wherein communicating the alarm further includes providing additional information on the physical location of the patient.

23. The method of claim 13, further comprising performing self checking/monitoring and reporting.
